(19) United States
(12) Patent Application Publication
(10) Pub. No.: US 2003/0068807 A1
(43) Pub. Date: Apr. 10, 2003

(54) Precursor conversion screening methods

(75) Inventors: James Hoch, La Jolla, CA (US); Veronique Dartois, San Diego, CA (US)

Correspondence Address: FOLEY & LARDNER, P.O. BOX 80278, SAN DIEGO, CA, 92138-0278, US

(73) Assignee: Microgenomics, Inc.

(21) Appl. No.: 10/210,792

(22) Filed: Jul. 31, 2002

Related U.S. Application Data
(60) Provisional application No. 60/309,503, filed on Aug. 1, 2001.

(51) Int. Cl. C12Q 1/68; C12N 7/00; C12N 7/01; C12N 7/02; C12N 5/00; C12N 5/02

(52) U.S. Cl. 435/235.1; 435/325; 435/239; 435/6

(57) ABSTRACT

The invention provides methods of identifying a host cell that encodes a metabolic pathway that converts a precursor molecule into a growth inhibitory compound, by: (a) culturing a population of host cells under conditions that allow expression of the metabolic pathway; (b) contacting the host cells, or an extract thereof, with a population of target cells and the precursor molecule; and (c) identifying a host cell that inhibits growth of the target cells in the presence, but not in the absence, of the precursor molecule, where an identified host cell from step (c) contains a metabolic pathway that converts the precursor molecule into a growth inhibitory compound. Methods of identifying host cells that encode a metabolic pathway that converts a precursor molecule into a desired product compound are also provided. Further provided are methods of identifying nucleic acid molecules encoding the metabolic pathways.

This application is a continuation-in-part of U.S. Application Serial No. 60/309,503, filed Aug. 1, 2001.

### FIELD OF THE INVENTION

The present invention relates to identifying host cells having useful metabolic pathways, and nucleic acids coding for the pathways.

### BACKGROUND OF THE INVENTION

The development of drug resistant strains of micro-organisms is a serious health concern. It is currently estimated that within the next 10 years, virtually all antibiotics currently employed for treating bacterial infections will no longer be effective, due to microbial resistance. New therapeutic compounds are thus urgently needed to meet the threat of drug resistant bacteria. Effective therapeutic compounds that combat diseases caused by viral and fungal pathogens are also needed.

Cancer is one of the leading causes of death in industrialized nations. Immune pathologies, including autoimmune pathologies, sepsis and chronic inflammation are also serious health concerns. New therapeutic compounds that inhibit cellular proliferation and pathogenic immune responses are thus urgently needed to treat these diseases.

The search for new therapeutic compounds, including anti-infectives, anti-cancer compounds and anti-inflammatory compounds, has traditionally been performed by screening collections of natural products and synthetic chemicals for a biological effect. From a promising lead compound, a library of related molecules can then be chemically synthesized and tested for increased efficacy and specificity, reduced toxicity and other desirable biological properties.

More recent drug discovery programs have employed mechanism-based approaches. Once a molecular target is identified, assays are designed to identify lead compounds that interact at a molecular level with the target. As with traditional approaches, structurally related compounds are then synthesized by combinatorial chemistry-based methods and screened for improved biological properties.

Strategies for discovering and producing therapeutic compounds from living organisms, and particularly microorganisms, have also been proposed. Generally, such strategies require that the organism-expresses an entire multi-step metabolic pathway for the biosynthesis of a compound with a desired therapeutic activity. However, it is a significant problem to clone and effectively reconstruct an entire biological pathway in order to produce a pharmaceutical compound on an industrial scale. Furthermore, such methods require that the therapeutic be a product produced in nature, thereby limiting the possibilities for therapeutic compounds.

The present invention provides improved methods of producing arid screening for therapeutic compounds that overcome the disadvantages of conventional methods, and provides related advantages as well.

### SUMMARY OF THE INVENTION

The present invention features methods of detecting a biochemical pathway able to convert a precursor compound into a desired product compound, such as a growth inhibitory compound. The desired product compound can be detected by standard methodology and is derived from the precursor compound.

Therefore, the invention provides a method of identifying a host cell that encodes a metabolic pathway that converts a precursor molecule into a growth inhibitory compound, where steps (a) and (b) are optional, by: (a) culturing a population of host cells under conditions that allow expression of the metabolic pathway; (b) contacting the host cells, or an extract thereof, with a population of target cells and the precursor molecule; and (c) identifying a host cell that inhibits growth of the target cells in the presence, but not in the absence, of the precursor molecule; where an identified host cell from step (c) contains a metabolic pathway that converts the precursor molecule into a growth inhibitory compound. In preferred embodiments a library of expressible nucleic acid molecules is introduced into the population of host cells prior to step (a). In various embodiments the growth inhibitory compound is an anti-infective compound, an anti-cancer compound, or an anti-inflammatory compound. In various embodiments the target cell is a bacterial cell, a fungal cell (e.g., from the genus Candida), a virus-infected cell, or a mammalian cell. In embodiments where the target cell is a bacterial cell, the cell can be a Staphylococcus aureus, an MRSA (methicillin resistant S. aureus), an Enterococcus faecium, a VRE (vancomycin resistant Enterococcus), a Streptococcus pneumoniae, a Salmonella typhi, a E. coli 0157, a Mycobacterium marinum or a Mycobacterium tuberculosis.

In addition, the invention provides methods of identifying a host cell that encodes a metabolic pathway that converts a precursor molecule into a desired product compound, where steps (a) and (b) are optional, by (a) culturing a population of host cells under conditions that allow expression of the metabolic pathway; (b) assaying the host cells, or extract thereof, for the presence of the desired product compound; and (c) identifying a host cell that contains the desired product compound in the presence, but not in the absence, of the precursor molecule; where an identified host cell from step (c) contains a metabolic pathway that converts the precursor molecule into a desired product compound. In various embodiments the assay can be an enzymatic assay, a binding assay, a reporter gene assay, a signaling assay and a growth inhibition assay. In preferred embodiments a library of expressible nucleic acid molecules is introduced into the population of host cells prior to step (a). In preferred embodiments, the nucleic acid molecules are derived from an environmental source, such as mud, soil, water, sewage, flood control channels, or sand. In various embodiments the host cell is a bacterial cell, a fungal cell, or a mammalian cell, and can be derived from an environmental source. In preferred embodiments, the precursor molecule is a drug-relevant pharmacophore molecule, a polyketide, an aminoglycoside, a β-lactam, a cyclosporin, a glycopeptide, a lipopeptide, a lipodepsipeptide, an azole, a triazole, an echinocandins, a pneumocandin, a macrolide, an azolide, a sufonamide, a tetracycline, a quinolone, oxazolidinone, a cationic peptide, or a cephem. In the most preferred embodiments the precursor molecule is 7-aminocephalosporanic acid (7-ACA), tetracycline, vancomycin, methicillin, fluconazole, or voriconazole. Where the culturing step is used in the above methods, the host cells are preferably cultured in the presence of a sub-lethal dose of the precursor molecule. In some embodiments the compound is isolated from an extract of the host cell.

In another aspect the present invention provides methods and selection strategies for identifying a nucleic acid molecule encoding a metabolic pathway that converts a precursor molecule to a desired product compound. The methods and selection strategies involve (a) providing a cell that contains a stress-responsive promoter fused to a gene essential for growth of said cell; (b) introducing a library of expressible nucleic acid molecules into a population of said cells; (c) culturing said cells under conditions that allow expression of said nucleic acid molecules; (d) contacting said cells with a sub-lethal dose of said precursor molecule and under conditions where the product of said essential gene is required for survival of said cells; and (e) identifying cells that survive in the presence but not in the absence of said precursor molecule, where the identified cell from step (e) contains a nucleic acid molecule that encodes a metabolic pathway that converts a precursor molecule into a desired product compound.

The stress-responsive promoter is “fused” to the gene when it is covalently attached in a manner that allows the promoter to control transcription of the coding region of the gene. A gene is “essential” for the growth of a cell when the cell is unable to grow to a colony visible with the unaided eye without the gene within 48 hours. “Stress-responsive” promoters are those that are activated under conditions of stress, such as heat, cold, and growth limiting nutrient availability. For example, a promoter that is turned on in response to a decreasing growth rate of a cell due to the presence of a compound that is toxic to the cell is a stress-responsive promoter. A compound is “toxic” to the cell when the presence of the compound causes the doubling time of the cell to increase by at least two-fold. The doubling time is the time necessary for the number of cells to double. A “sub-lethal” dose is one that allows for a substantially normal growth rate. Sub-lethal doses increase the doubling time of the cell by less than two-fold.

The summary of the invention described above is not limiting and other features and advantages of the invention will be apparent from the following detailed description of the preferred embodiments, as well as from the claims.

### DETAILED DESCRIPTION OF THE INVENTION

The present invention takes advantage of the fact that many important pharmaceuticals are structural derivatives of well-characterized precursor molecules, such as lead compounds identified by conventional pharmacological approaches. These precursor molecules may themselves be inactive or only weakly active, or have undesirable biological properties such as toxicity or unpleasant side-effects in mammals. However, such molecules are known or predicted to form the backbone of classes of important pharmaceuticals.

The inventors have discovered that organisms can be identified that contain enzymatic pathways that convert pharmaceutical precursor molecules into therapeutically active compounds. According to the invention methods, the genes that encode the conversion pathways can be cloned. Alternatively or additionally, the invention methods can be used to isolate and characterize these therapeutically active compounds from the organism or recombinant organism.

By starting from a known precursor molecule, there is a high likelihood of identifying novel derivative compounds with desirable biological properties, which can be rapidly isolated and characterized. Additionally, by starting with a precursor near the end of a synthetic pathway, rather than requiring that the organism biosynthesize an entire active compound de novo, therapeutic compounds not normally found in nature can be identified. As a further advantage, by requiring the microorganism only to perform a limited number of biosynthetic steps, it is also simpler to clone and reconstruct pathways for bioproduction of the therapeutic compounds from the precursor. Furthermore, as opposed to making and testing derivatives of lead compounds by chemical methods, the invention method is faster, and not limited by technical considerations that limit combinatorial chemistry approaches.

As used herein, the term “precursor molecule” is intended to mean a chemical structure which can be converted into another, structurally related, chemical structure or derivative. A precursor molecule can be a chemical structure such as a pharmaceutical or pharmacophore, or a chemical structure such as a macromolecule composed of nucleic acids or amino acids. For example, a precursor molecule can be a drug-relevant pharmacophore which is a basic backbone or scaffold structure of a pharmaceutical. Exemplary precursor molecules include polyketides, aminoglycosides, β-lactams, cyclosporins, glycopeptides, lipopeptides, lipodepsipeptides, azoles, triazoles, echinocandins, pneumocandins, macrolides, azolides, macrolides, azolides, sufonamides, tetracyclines, quinolones, oxazolidinone, cationic peptides, and cephems. For example, a precursor molecule can be 7-aminocephalosporanic acid (7-ACA), tetracycline, vancomycin, methicillin, fluconazole, and voriconazole.

One of skill in the art will understand that a precursor molecule used in the methods of the invention will not be lethal to the host cell. For example, the precursor molecule can be inactive, or it can be used at a concentration determined to be sub-lethal to the particular host cell used in the methods of the invention.

As used herein the term “desired product compound” is intended to mean a chemical structure, derived from a precursor molecule, having a desired function. A desired product compound is a derivative of the precursor molecule and will therefore have some structures in common with the precursor molecule as well as one or several structures that differ from the precursor molecule. For example, a desired product compound may have one or several functional groups that are different from the precursor molecule. However, one of skill in the art will recognize that the desired product compound is derived from the precursor molecule.

The desired product compound is selected based on a desired function. A desired function is any therapeutically relevant function for which an assay is known or can be designed. A desired function can be, for example, growth inhibition including the inhibition of bacterial, viral, and fungal growth. In addition, a desired function can be, for example, inhibition or enhancement of binding to a receptor or other protein, inhibition or enhancement of an enzymatic reaction, or any other assayable function.

This desired function can be the direct result of the desired product compound or can be an indirect result of the desired product compound. For example, a desired product compound can be an active anti-infective compound, or a compound that does not have anti-infective activity on its own, but potentiates the synthesis of a compound with anti-infective activity. In addition, for example, the desired product compound can potentiate the activity of a compound already present in the cell by cooperative enhancement or synergy. Other examples of indirect action by a desired product compound include the inhibition of the conversion of a normal or induced metabolite to a non-active compound, induction of secondary metabolism pathways, and enhancing the secretion of end products with the desired activity.

A desired product compound that inhibits growth is referred to herein as a “growth inhibitory compound.” Several assays can be used to determine growth or proliferation of cells. For example, a zone of clearing in a lawn of bacteria can be used to indicate growth inhibition (see Example I). In addition, the growth of bacteria or other cells in liquid culture can be determined by optical methods, for example, determining the optical density of a solution of bacterial cells at a wavelength of 600 nm in a spectrophotometer. Further examples include the use of an oxygen sensing method, for example the fluorescence quenching system developed by Beckton-Dickinson. The growth of mammalian cells can be determined using viability dyes such as trypan blue or Alamar Blue, or using functional assays such as a lactose dehydrogenase (LDH) assay, ³H thymidine uptake assay, or a 3-(4,5-dimethylthiazol-2-yl)- 2,5,-diphenyl tetrazolium bromide (MTT) assay. Growth is inhibited when the presence of at least 25% fewer cells is detectable in the presence of the compound versus the absence of the compound.

Exemplary desired product compounds are anti-infective compounds, anti-tumor compounds and anti-inflammatory compounds. However, one of skill in the art will recognize that the methods of the invention can be applied to select for product compounds with any desired functional activity for which an assay exists or can be designed. Advantageously, a desired product compound can have, for example, greater growth-inhibitory activity, improved synthesis, and less toxic side-effects. In addition, one of skill in the art will recognize that the structure of the desired product compound does not need to be known a priori since it is selected on the basis of its function. In the methods of the invention, a host cell performs the chemical conversion of the precursor molecule, and so no bias is introduced into the design of the product compound.

The term “host cell” as used herein is intended to mean a cell with a metabolic pathway that converts a precursor molecule to a desired product compound. A host cell can be, for example, a bacterial cell, a yeast or other fungal cell, or a mammalian cell. A host cell can be derived from any source including, for example, from an environmental source such as mud, soil, water, sewage, flood control channels, and sand. In addition, a host cell can be a cell that is non-culturable, or is not easily cultured, in the laboratory as well as cells that are culturable. For example, DNA can be extracted from a non-culturable cell from an environmental source and this DNA can be used to generate a library in a culturable organism for use in the methods of the invention. When a library of expressible nucleic acids is introduced into the host cell, it is understood that the host cell is able to be transformed or transfected with genetic material.

As used herein the term “metabolic pathway” is intended to mean an enzymatic pathway that exists in a cell that catalyzes the conversion of a starting material to a product. A metabolic pathway can consist of a single enzyme or a series of several enzymes that catalyze the conversion of a starting material to a product. A metabolic pathway includes the enzymes, and the genes that encode them, that catalyze the conversion of the starting material to a product.

As used herein the term “target cell” is intended to mean a cell for which growth inhibition is desired. Target cells can be, for-example, bacterial cells, fungal cells, virus-infected cells, and mammalian cells such as tumor cells and immune cells. Exemplary target bacterial cells include Staphylococcus aureus, MRSA (methicillin resistant S. aureus), Enterococcus faecium, VRE (vancomycin resistant Enterococcus), Streptococcus pneumoniae, Salmonella typhi, E. coli 0157, Mycobacterium marinum and Mycobacterium tuberculosis. Fungal cells include, for example, any cell from the genus Candida. Several mammalian cells are available that are permissive for infection by certain viruses. These cells include, for example, HeLa cells, COS7 cells, and CHO cells.

As used herein, the term “cell-free extract” is intended to mean a mixture of cellular components that does not include intact cells. For example, cells can be lysed and the contents of the cell collected. Several protocols are well known in the art for collecting particular fractions of a cell, including, for example, a nuclear extract, a membrane preparation, or a cytoplasmic extract. In addition, a cell-free extract can be, or include, culture media in which the cells are growing (sometimes called conditioned media). This culture media can contain several proteins that have been secreted by the cells in culture.

As used herein, the term “nucleic acid” is intended to mean a single- or double-stranded DNA or RNA molecule including, for example, genomic DNA, cDNA and mRNA. The term is intended to include nucleic acid molecules of both synthetic and natural origin and can represent either the sense or antisense strand, or both, of a native nucleic acid molecule. Nucleic acids useful in the invention include, for example, mutagenized DNA, environmental DNA, combinatorial libraries, and recombinant DNA. Mutagenized DNA can be the result of mutagenesis by a process including, for example, random, chemical, PCR-based, and directed mutagenesis. In addition, environmental DNA can be derived, for example, from mud, soil, water, sewage, flood control channels, and sand (see, for example, table I). Soil sources include, for example, forest soil, cultivated or garden soil, marsh or swamp soil, desert soil, terrestrial and marine sediments.

As used herein the term “library” is intended to mean a library of expressible nucleic acid molecules. A library is collection of genetic material from an organism and can include for example, genomic DNA or cDNA. Genes present in the genetic material are operably associated with regulatory regions that drive expression of the genes in an appropriate library host organism. The term “operably-associated” refers to an association in which the regulatory regions and the DNA sequence to be expressed are joined and positioned in such a way as to permit transcription, and ultimately, translation. Libraries useful in the invention include for example, libraries generated from mutagenized DNA, environmental DNA, and recombinant DNA (see, for example, table I). In addition, combinatorial libraries can be used in the invention methods.

TABLE I
Environmental Libraries

| | | #CLONES | |
|---|---|---|---|
| | | Screened | |
| SOURCE | Generated | S. aureus | Fungi |
| SAN DIEGO Lake | 2.10⁶ | 300,000 | 480,000 |
| ROME, dry soil | 1.10⁶ | 100,000 | 140,000 |
| BAHAMAS | | | |
| Mangrove | 150,000 | | 150,000 |
| Coastal | 130,000 | 60,000 | |
| Coppice | | | |
| Oyster Pond | 12,500 | 12,500 | |
| Light House | 120,000 | 50,000 | |
| Cave (Gram+) | | | |
| BELGIUM, river | 400,000 | 70,000 | 155,000 |
| bed | | | |
| JAPAN, | 750,000 | 150,000 | 260,000 |
| cultivated soil | | | |
| Activated | 350,000 | 110,000 | 100,000 |
| Sludge, TN | | | |
| NORMALIZED-400 | 1.10⁶ | 40,000 | 50,000 |
| Joshua Tree | 3.10⁶ | | 100,000 |
| Natl Park, | | | |
| dry soil | | | |
| COMPOSITE | 3.10⁶ | 70,000 | 100,000 |
| BAJA | 200,000 | 60,000 | 100,000 |
| CALIFORNIA, | | | |
| Cactus Field | | | |
| MARINE ARCHEA | 60,000 | 60,000 | |
| MARINE | 250,000 | 60,000 | 100,000 |
| ACTINOMYCETES | | | |

Libraries useful in the invention are described herein and in PCT application number W00022170 and in U.S. Pat. No. 5,824,485, both of which are incorporated herein by reference. Briefly, the methods of the invention include providing genetic material derived from one or more organisms of interest, manipulating the genetic material, and introducing the genetic material into a host organism via a cloning or expression vector so that one or more genes of the organisms of interest are transferred to and expressed in the library host organism. The library host organisms containing donor genetic material are pooled to form a library.

The transferred genetic material, typically comprises a random assortment of genes, the expression of which is driven and controlled by one or more functional regulatory regions. The expression construct or vector can provide some of these regulatory regions. The genes of the organisms of interest are transcribed, translated and processed in the library host organism to produce functional proteins.

As stated herein, the organisms of interest can be derived from several sources including, for example, environmental sources. These organisms may or may not be cultivable with current state-of-the-art microbiological techniques. Since only a minority of the microbes found in nature can be cultured in the laboratory, an advantage of the present invention is that the organism does not have to be cultivable to be utilized herein (Torsvik et al. 1990, Appl Env Micro, 56:782-787)

Nucleic acids can be isolated from organisms of interest by a variety of methods known in the art to obtain high quality nucleic acids that are free of nicks, single stranded gaps, and partial denaturation, and are of high molecular weight (especially for genomic DNA cloning), in order to construct gene expression libraries that are fully representative of the genetic information of donor organisms. To prepare high quality nucleic acid, the organisms are lysed and nucleases or other degradative proteins are inactivated. A number of standard cell lysis techniques can be used, including freezing in liquid nitrogen, grinding in the presence of glass or other disruptive agents, as well as simple mechanical shearing or enzymatic digestion. RNA isolated from donor organisms can be converted into complementary DNA (cDNA) using reverse transcriptase. In addition, damaged DNA can be repaired in vitro prior to cloning, using enzymatic reactions commonly employed during second strand synthesis of complementary DNA (Sambrook et al. 1989, in “Molecular Cloning” 2nd Edition)

When preparing an expression library from DNA, if the quantity is low after extraction (<100 ng), the DNA can be first ligated into a high-efficiency cloning system, for example, SuperCos. The inserts in the clones are amplified and are released from the vector by restriction enzyme digestion. If sufficient amount of original DNA sample is available, or if the DNA has been amplified, the DNA can be ligated directly into an expression vector. Exemplary vectors include, for example, plasmids; cosmids; phagemids; artificial chromosomes, such as yeast artificial chromosomes (YACs), and bacterial artificial chromosomes (BACs, Shizuya et al. 1992, Pro Natl Cad Sci 89: 8794-8797) or modified viruses, but the vector must be compatible with the library host organism. Useful vectors include, for example, lambda gtll, SuperCosl (Stratagene), pBluescript (Stratagene), CDM8, pJB8, pYAC3, pYAC4 (see Current Protocols in Molecular Biology, 1988, Ed. Ausubel et al., Greene Publish. Assoc. & Wiley Interscience, which is incorporated herein by reference)

An expression vector can contain selectable or screenable marker genes for initially isolating, identifying or tracking host organisms that contain donor DNA. The expression vector can also contain sequences that permit maintenance and/or replication of the vector in one or more host organism, or integration of the vector into the host chromosome. In addition, it can be advantageous to use shuttle vectors which can be replicated and maintained in at least two host organisms, such as, for example, bacteria and mammalian cells, bacteria and yeasts, bacteria and plant cells, or gram positive and gram negative bacteria.

In yeast, a number of vectors containing constitutive or inducible promoters can be used with Saccharomyces cerevisiae (baker's yeast), Schizosaccharomyces pombe (fission yeast), Pichia pastoris, and Hansenula polymorpha (methylotropic yeasts). In addition, a variety of mammalian expression vectors are commercially available. Further, a number of viral-based expression systems, such as adenovirus and retroviruses, can be utilized.

Prior to insertion into a vacant expression vector, DNA inserts can be separated according to size by standard techniques, including but not limited to, agarose gel electrophoresis, dynamic density gradient centrifugation, and column chromatography. In addition, DNA can be pre-selected for a specific property, such as certain DNA sequences by first hybridizing the DNA to nucleic acid probes containing these sequences. The insertion into an expression vector can be accomplished by ligating the DNA fragment into an expression vector which has complementary cohesive termini. Any restriction site desired can be produced by ligating nucleotide sequences such as linkers or adaptors onto the DNA termini.

Expression constructs are then introduced into the appropriate library host organisms. A variety of methods can be used, which include, for example, transformation, transfection, infection, conjugation, protoplast fusion, liposome-mediated transfer, electroporation, microinjection and microprojectile bombardment. Exemplary prokaryotic library host organisms can include, for example, Escherichia coli, Bacillus subtilis, Streptomyces lividans, Streptomyces coelicolor. Yeast species such as Saccharomyces cerevisiae, Schizosaccharomyces pombe, and Pichia pastoris, and Hansenula polymorpha (methylotropic yeasts) can also be used. In addition filamentous ascomycetes, such as Neurospora crassa and Aspergillus nidulans, and plant cells such as those derived from Nicotiana and Arabidopsis can be used. Preferred mammalian library host cells include but are not limited to those derived from humans, monkeys and rodents, such as chinese hamster ovary (CHO) cells, NIH/3T3, COS, 293, VERO, etc (see Kriegler N. in “Gene Transfer and Expression: A Laboratory Manual”, New York, Freeman & Co. 1990). After the library host cells containing expression constructs are pooled to form a library, they can be optionally amplified by techniques known in the art.

Several assays can be utilized in the methods of the invention in order to select for a desired product compound. For example, growth inhibition assays, as described above, can be used to screen for a growth inhibitory compound of the invention. If a product compound is desired that does not affect growth, but affects a different pathway in a cell, an assay that detects the desired function can be used. For example, many high-throughput enzymatic and binding assays that can detect a myriad of different cellular functions and pathways are known to those skilled in the art.

The invention provides methods where a desired product compound is not a growth inhibition compound. For example, if a high affinity G-protein coupled receptor (GPCR) ligand is desired, one can take an inactive or weakly active precursor molecule and use the methods of the invention to select for the desired high affinity ligand product compound. An inactive or weakly active precursor molecule can be obtained, for example, from an initial high through-put screening (HTS) receptor binding assay. This precursor molecule can be added to different cultures of host cells and after a certain amount of time, for example, two days, a cell-free extract can be made from the host cells. These extracts can be assayed for receptor binding activity using the HTS receptor binding assay or other relevant binding assay. If an extract is found that contains a high affinity ligand, several options are available. For example, the high affinity ligand can be isolated from the extract using conventional purification procedures as described herein. In addition, the DNA from the host cell that produced the active extract can be used to generate a library from which a gene or genes responsible for the conversion of the inactive precursor to the active product can be cloned.

One having skill in the art will understand/that any functional assay format in addition to the HTS receptor binding assay exemplified above can be used. For example, co-immunoprecipitation assays and transcription based assays such as reporter assays and two-hybrid assays can be. Such assays are well known in the art and can be found in standard reference texts such as Sambrook et al., supra, and Ausubel et al., supra, 1999. Additional methods include, for example, scintillation proximity assay (SPA) (Alouani, Methods Mol. Biol. 138:135-41 (2000)), UV or chemical cross-linking (Fancy, Curr. Opin. Chem. Biol. 4:28-33 (2000)), competition binding assays (Yamamura et al., Methods in Neurotransmitter Receptor Analysis, Raven Press, New York, 1990), biomolecular interaction analysis (BIA) (Weinberger et al., Pharmacogenomics 1:395-416 (2000)), mass spectrometry (MS) (McLafferty et al., Science 284:1289-1290 (1999) and Degterev, et al., Nature Cell Biology 3:173-182 (2001)), nuclear magnetic resonance (NMR) (Shuker etal., Science 274:1531-1534 (1996), Hajduk et al., J. Med. Chem. 42:2315-2317 (1999), and Chen and Shapiro, Anal. Chem. 71:669A-675A (1999)), and fluorescence polarization assays (FPA) (Degterev et al., supra, 2001) which are incorporated herein by reference.

Growth inhibitory compounds including, for example, anti-infective, anti-tumor and anti-inflammatory compounds can be produced by methods of the invention. Anti-infective compounds include, for example, anti-bacterial anti-viral and anti-fungal compounds. An example of a screen for an anti-bacterial compound can be found in Example I below. Anti-viral compounds can be found in a similar manner with some modifications. The invention provides a method of identifying a host cell that encodes a metabolic pathway that converts a precursor molecule into an anti-viral compound, by: (a) culturing a population of host cells under conditions that allow expression of the metabolic pathway; (b) contacting the host cells, or an extract thereof, with a population of virus-infected target cells and the precursor molecule; and (c) identifying a host cell that inhibits growth of the target cells in the presence, but not in the absence, of the precursor molecule. This method is useful for viruses that do not cause a cytotoxic or lytic infection. The method would identify a product compound that could kill a virus infected cell. In some cases this is desirable, however in other cases it is desirable to kill the virus without killing the cell that is infected with the virus. When a cell is infected with a lytic virus and it is desirable to kill the virus without killing the cell, step (c) in the method described above can be modified so as to identify a host cell that allows growth of the target cell in the presence, but not in the absence, of the precursor molecule. One of skill in the art will understand that with this method and others control experiments will also be performed. For example, in the last scenario a compound found by this modified method would also be tested again an un-infected version of the target cell to determine whether the product compound is acting selectively against the virus.

If necessary, a desired product compound can be isolated from the cellular mixture. Several procedures are known to those skilled in the art for the isolation of chemical compounds from a mixture including a cellular mixture. Classically, a chemical compound of interest is purified to homogeneity by sequential fractionation and assay cycles of the specific activity of interest. A first step in an isolation procedure is to prepare a cell-free extract as described above. Steps can be taken to preserve the structure of the compound of interest, for example, if the compound is a protein the sample can be prepared at 4° C. and protease inhibitors can be included in the sample preparation.

Fractionation of the sample can be performed using any separation procedure including for example, precipitation, density gradients, and chromatography. Column chromatography can be performed efficiently using an HPLC or FPLC. Several column matrix materials are available that contain different charge groups and other properties for binding the compound of choice. A possible advantage of the methods of the invention is that while the structure of the desired product compound is not known, the structure of the precursor molecule is known and because the product is related to the precursor it can aid in designing an isolation protocol. For example, if a precursor compound is known to be highly acidic, one could chose a basic charge column matrix as a first step for purification of the product. In some cases changes to the product compound compared to the precursor compound can result in a significantly different isolation profile, however knowledge of the structure of the precursor compound can provide guidance in the design of an isolation protocol. Different fractions obtained from the separation procedure are subsequently assayed for the presence of the product compound. One or more cycles of separation followed by assay of the fractions can be required.

In addition, to purification of the product compound, a gene from the host cell that encodes a protein that converts a precursor molecule to desired product compound can be isolated. In the methods of the invention where a host cell has been transformed with a library of expressible nucleic acid molecules, a gene of interest can be cloned by utilizing aspects of the library design. For example, if the library expression vector has an artificial sequence such as a tag sequence on both sides of the cloning site of the genes, one can use the tag to isolate the gene. For example, DNA primers that are complementary to the tag sequence can be used to amplify the intervening gene using the polymerase chain reaction (PCR)

In order to clone a gene from a host cell that does not contain a library, one can isolate the DNA from the host cell and generate a library as described herein. This library can then be transformed into an appropriate library host cell and colonies from this library can be assayed for the desired function. Once a positive colony is found, the gene of interest can be cloned using the procedure described above.

It is possible that when screening for a desired function, such as growth inhibition, a host cell is found that inhibits growth of the target cell and no conversion of the precursor molecule is detected. An example of this can be found in isolate 1-6 in Example I. In this case, the host cell can still be useful for identifying a drug target. For example, if the host cell was transformed with an expression library, the gene can be cloned and that gene can represent a target in a pathway involved indirectly in growth inhibition.

In another embodiment, the invention provides a method of identifying a nucleic acid molecule encoding a metabolic pathway that converts a precursor molecule to a desired product compound, by: (a) providing a cell that contains a stress-responsive promoter fused to a gene essential for growth of the cell; (b) introducing a library of expressible nucleic acid molecules into a population of the cells; (c) culturing the cells under conditions that allow expression of the nucleic acid molecules; (d) contacting the cells with a sub-lethal dose of the precursor molecule and under conditions where the product of the essential gene is required for survival of said cells; and (e) identifying cells that survive in the presence but not in the absence of the precursor molecule where an identified cell from step (e) contains a nucleic acid molecule that encodes a metabolic pathway that converts a precursor molecule into a desired product compound.

Stress responsive promoters are promoter sequences that have been found to respond to stresses on the cell or to slow growth from the cell. Exemplary stress responsive promoters include, for example, uspA, grpE, katG, micF, and trxA (see Israel et al., App. Envir. Micro. 64:4346-4352 (1998)) incorporated herein by reference. Essential genes that can be used in this method include, for example, essential amino acid biosynthetic genes. Exemplary essential genes include thymidylate synthase (thyAB), RNA polymerase (rpoB and rpoC) and chorismate synthetase.

It is understood that modifications which do not substantially affect the activity of the various embodiments of this invention are also included within the definition of the invention provided herein. Accordingly, the following examples are intended to illustrate but not limit the present invention.

### EXAMPLE I

This example shows a method of identifying a cell expressing a metabolic pathway that converts the precursor molecule 7-aminocephalosporanic acid (7-ACA) into an anti-infective compound effective against the target organism Bacillus subtilis. This example further shows alternative embodiments of the method, in which the host organisms are either environmental isolates or E. coli transfected with genomic libraries.

For these experiments, 7-ACA was purchased from Sigma-Aldrich. The sublethal concentration for B. subtilis was determined to be between 50 and 100 μg/ml.

Genomic libraries from 400 terrestrial culturables collected at various locations through out the United States were constructed in SuperCosl as the vector and E. coli as the host cell, and plated on LB supplemented with 30 μg/ml kanamycin and 50 μg/ml 7-ACA at a cell density which provided 500 clones per Petri dish.

Alternatively, culturable environmental isolates of marine microorganisms obtained from marine symbiants collected in the Bahamas and Baja California were replicated in duplicates from 96-well storage plates onto Glycerol Artificial Sea Water (GASWA) medium plates supplemented with 50 μg/ml 7-ACA.

The plates were incubated for 2 days at 30° C. to allow host cell colony formation. Growth of host cells was arrested by UV exposure (2 min for library clones and up to 4 h for environmental isolates). Each plate containing library clones was overlayed with 3 ml of TSB (Tryptic Soy Broth) soft agar (0.7%) containing 10⁶ B. subtilis target cells (-5 μg/ml of a stationary phase culture) and supplemented with 7-ACA (50 μg/ml). Each copy of the plate containing 96 environmental isolates was overlayed with 8 ml of TSB soft agar containing 3×10⁶ B. subtilis cells with and without 7-ACA.

Library clones which gave a clearing zone or halo in the presence of 7-ACA were recovered and tested against B. subtilis in the absence of 7-ACA. Only those clones which inhibited growth of B. subtilis in a 7-ACA-dependent manner were retained for further analysis. Likewise, environmental isolates which exhibited 7-ACA-dependent inhibition of B. subtilis were retained as positive candidates for 7-ACA backbone conversion.

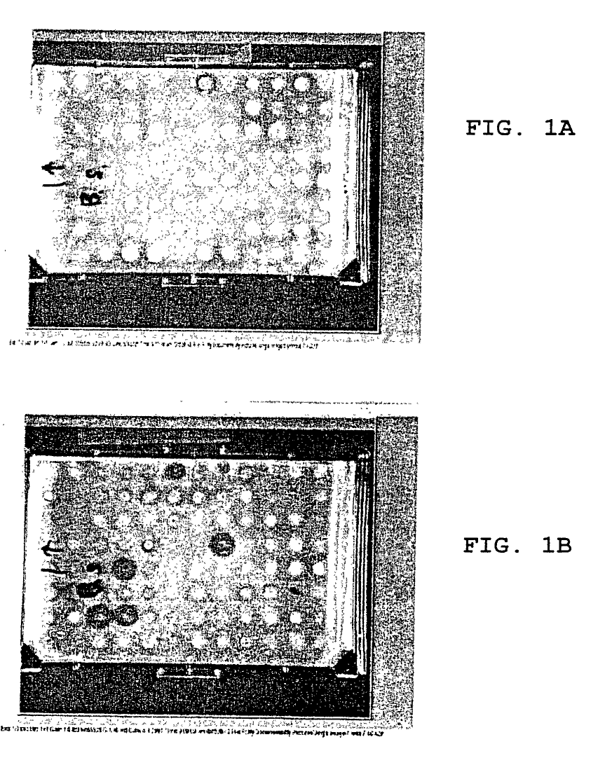

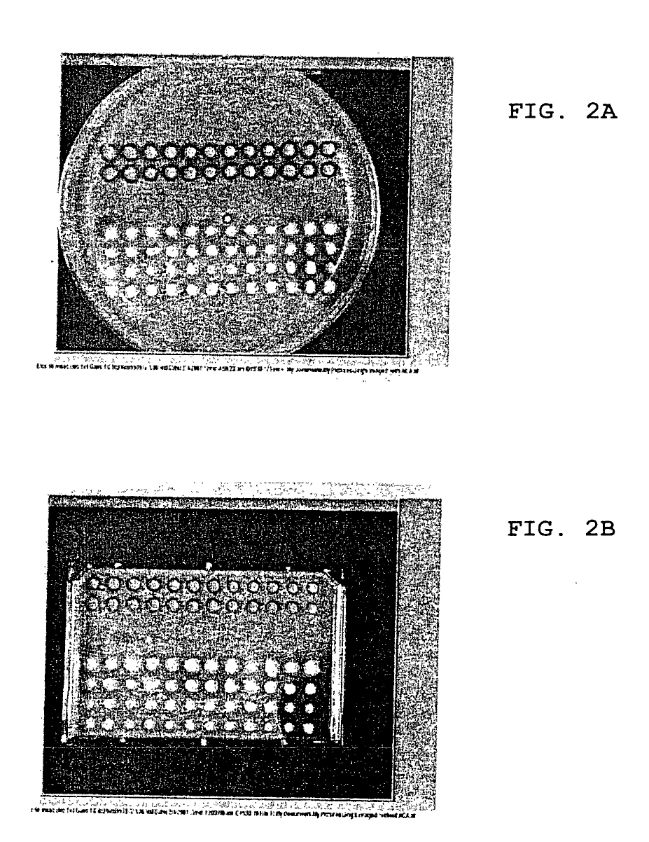

An example of the results of the method described above using E. coli transfected with an environmental genomic library as the host cells is shown in FIG. 1, and using environmental isolates of marine microorganisms as the host cells is shown in FIG. 2. In both figures the top panel (A) shows a plate without 7-ACA, whereas the bottom panel (B) shows a plate with 7-ACA.

As shown in FIG. 1, the top two rows of E. coli recombinant clones exhibited increased growth inhibition of B. subtilis in the presence of 7-ACA. As shown in FIG. 2, marine isolates identified by the following row#-column# exhibited increased growth inhibition of B. subtilis in the presence of 7-ACA: isolates 1-6, 2-5, 2-6, 2-7, 4-8, 5-4, 6-3 and 6-4. Further characterization of isolate 1-6 showed that it produces a protein that acts in conjunction with 7-ACA to inhibit growth of B. subtilis, rather than directly converting 7-ACA into an anti-infective.

### EXAMPLE II

This example shows a method of preparing Glycerol Artificial Sea Water (GASWA) agar plates suitable for culturing isolates of marine organisms.

Briefly, to prepare 1 L of medium, the following ingredients were added to 500 ml ddH₂O in a 2 L flask while stirring.

NaCl 20.8 g

KCl 0.6 g

MgSO₄4 4.8 g

MgCl₂.6H₂O 4.0 g

K₂HPO₄ 0.009 g

FeSO₄7H₂₀ 0.008 g

INSTANT OCEAN® (Aquarium Systems, Mentor, Ohio) 2.0 g (from pet store Aquarium section)

Tris—0.5 g

Peptone 4.09

Yeast Extract 2.0 g

Agar 20.0 g

INSTANT OCEAN® is a commercially available synthetic aquarium salt and contains every necessary major, minor, and trace element and has no nitrates and no phosphates. Any equivalent salt mixture can be substituted.

When the ingredients were mixed, approximately 500 ml of ddH₂O was added, to make a total volume of 1 L. The media was autoclaved for 1 hour, after which 6.6 ml of 30% glycerol was added while stirring. After the media cooled to approximately 55° C., plates were poured.

Throughout this application various publications have been referenced within parentheses. The disclosures of these publications in their entireties are hereby incorporated by reference in this application in order to more fully describe the state of the art, to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

What is claimed is:

1. A method of identifying a host cell that encodes a metabolic pathway that converts a precursor molecule into a growth inhibitory compound, comprising:
identifying a host cell or its extract that inhibits growth of said target cells in the presence, but not in the absence, of said precursor molecule;
wherein an identified host cell contains a metabolic pathway that converts said precursor molecule into a growth inhibitory compound.

2. The method of claim 1 further comprising culturing a population of the host cells under conditions that allow expression of the metabolic pathway; and
contacting the host cells, or an extract thereof, with a population of target cells and the precursor molecule.

3. The method of claim 1, wherein said growth inhibitory compound is selected from the group consisting of an anti-infective compound, an anti-cancer compound and an anti-inflammatory compound.

4. The method of claim 3, wherein said anti-infective compound is selected from the group consisting of an anti-bacterial compound, an anti-viral compound, and an anti-fungal compound.

5. The method of claim 1, wherein said target cell is selected from the group consisting of a bacterial cell, a fungal cell, a virus-infected cell and a mammalian cell.

6. The method of claim 5, wherein said target bacterial cell is selected from the group consisting of Staphylococcus aureus, MRSA (methicillin resistant S. aureus), Enterococcus faecium, VRE (vancomycin resistant Enterococcus), Streptococcus pneumoniae, Salmonella typhi, E. coli 0157, Mycobacterium marinum and Mycobacterium tuberculosis.

7. The method of claim 5, wherein said target fungal cell is from the genus Candida.

8. A method of identifying a host cell that encodes a metabolic pathway that converts a precursor molecule into a desired product compound, comprising:
identifying a host cell that contains said desired product compound in the presence, but not in the absence, of said precursor molecule;
wherein an identified host cell contains a metabolic pathway that converts said precursor molecule into a desired product compound.

8. The method of claim 8, further comprising assaying said host cells, or an extract thereof, for the presence of said desired product compound, and wherein said assay is selected from the group consisting of an enzymatic assay, a binding assay, a reporter gene assay, a signaling assay and a growth inhibition assay.

9. The method of claim 1 or 8, comprising introducing a library of expressible nucleic acid molecules into said population of host cells prior to said identifying step.

11. The method of claim 10, wherein said nucleic acid molecules are derived from an environmental source.

12. The method of claim 11, wherein said environmental source is selected from the group consisting of mud, soil, water, sewage, flood control channels, and sand.

13. The method of claim 1 or 8, wherein said host cell is selected from the group consisting of a bacterial cell, a fungal cell, and a mammalian cell.

14. The method of claim 13, wherein said bacterial cell is derived from an environmental source.

15. The method of claim 14, wherein said environmental source is selected from the group consisting of mud, soil, water, sewage, flood control channels, and sand.

16. The method of claim 1 or 8, wherein said precursor molecule is a drug-relevant pharmacophore molecule.

17. The method of claim 1 or 8, wherein said precursor molecule is selected from the group consisting of a polyketide, an aminoglycoside, a β-lactam, a cyclosporin, a glycopeptide, a lipopeptide, a tetracycline, a quinolone, a cationic peptide, and a cephem.

18. The method of claim 1 or 8, wherein said precursor molecule is selected from the group consisting of 7-aminocephalosporanic acid (7-ACA) , tetracycline, vancomycin, methicillin, and fluconazole.

19. The method of claim 1 or 8, wherein said host cells are cultured in the presence of a sub-lethal dose of said precursor molecule.

20. The method of claim 1 or 8, further comprising isolating said compound from said host cell or an extract of said host cell.

21. The method of claim 1 or 8, further comprising isolating a nucleic acid molecule or molecule from said host cell that encodes said metabolic pathway.

22. A method of identifying a nucleic acid molecule encoding a metabolic pathway that converts a precursor molecule to a desired product compound, comprising:
(a) providing a cell that contains a stress-responsive promoter fused to a gene essential for growth of said cell;
(b) introducing a library of expressible nucleic acid molecules into a population of said cells;
(c) culturing said cells under conditions that allow expression of said nucleic acid molecules;
(d) contacting said cells with a sub-lethal dose of said precursor molecule and under conditions where the product of said essential gene is required for survival of said cells; and
(e) identifying cells that survive in the presence but not in the absence of said precursor molecule;
wherein an identified cell from step (e) contains a nucleic acid molecule that encodes a metabolic pathway that converts a precursor molecule into a desired product compound.
